(19) United States
(12) Patent Application Publication
(10) Pub. No.: US 2014/0234955 A1
(43) Pub. Date: Aug. 21, 2014

(54) CELL CULTURE DEVICE

(71) Applicant: SAMSUNG ELECTRO-MECHANICS CO., LTD., Suwon (KR)

(72) Inventors: Bo Sung KU, Suwon (KR); Sang Jin KIM, Suwon (KR)

(73) Assignee: SAMSUNG ELECTRO-MECHANICS CO., LTD., Suwon (KR)

(21) Appl. No.: 13/866,821

(22) Filed: Apr. 19, 2013

(30) Foreign Application Priority Data
Feb. 19, 2013 (KR) 10-2013-0017408

(51) Int. Cl. C12M 1/34

(52) U.S. Cl. CPC C12M 41/44
USPC 435/297.2; 435/289.1

(57) ABSTRACT

There is provided a cell culture device including a cell chip receiving part coupled to a cell chip and having at least one passage through which a liquid-type medium is circulated, and a vortex generation part formed in the passage to generate a vortex in the liquid-type medium.

### CROSS-REFERENCE TO RELATED APPLICATIONS

This application claims the priority of Korean Patent Application No. 10-2013-0017408 filed on Feb. 19, 2013, in the Korean Intellectual Property Office, the disclosure of which is incorporated herein by reference.

### BACKGROUND OF THE INVENTION

1. Field of the Invention

The present invention relates to a cell culture device, and more particularly, to a cell culture device allowing a reaction of a cell to a liquid type medium to be inspected in an environment similar to that of the interior of a body.

2. Description of the Related Art

Demand for bio-medical devices and bio-technologies for promptly diagnosing various human diseases has recently increased. Accordingly, the development of experimental devices and instruments capable of providing diagnosis results in a relatively short time, as compared with existing, relatively time-consuming methods of diagnosing diseases in hospitals and laboratories has been actively conducted.

In order to develop new medicines and perform experiments to determine the stability of new medicines, it is essential to observe a reaction between a new medicine (i.e. a drug) and cells. In general, an experiment for determining a reaction between a drug and cells is performed by using a culture plate, or the like.

However, since a reaction between a drug and cells conducted in a culture plate may be quite different from a reaction between drug and cells occurring inside a body, it is difficult to exactly observe or inspect the reaction between a drug and cells based only on a result of an experiment using the culture plate. Therefore, there is a need to develop a new device allowing for reactions between drugs and cells to be observed in an environment similar to that of the interior of a body.

Meanwhile, as related art, there are provided Patent Documents 1 and 2 searched for by the present applicant. However, both of Patent Documents 1 and 2 relate to a polymerase chain reaction (PCR) and are not appropriate as technologies for solving the foregoing limitations.

### RELATED ART DOCUMENTS

(Patent Document 1) KR2005-117811 A

(Patent Document 2) JP2010-203779 A

### SUMMARY OF THE INVENTION

An aspect of the present invention provides a cell culture device allowing a reaction between a liquid type medium and cells to be observed or inspected in an environment similar to that of the interior of a body.

According to an aspect of the present invention, there is provided a cell culture device, including: a cell chip receiving part coupled to a cell chip and having at least one passage through which a liquid-type medium is circulated; and a vortex generation part formed in the passage to generate a vortex in the liquid-type medium.

The vortex generation part may include a plurality of projections formed on a side wall of the passage.

The plurality of projections may be formed at intervals in a height direction of the side wall.

The plurality of projections may be formed to have different heights in a length direction of the side wall.

The vortex generation part may include a plurality of projections formed on a bottom of the passage.

The plurality of projections may be formed at intervals in a width direction of the bottom.

The plurality of projections may be formed to have different heights in a length direction of the bottom.

The vortex generation part may include two or more types of projections having different heights.

The vortex generation part may have a cylindrical form or a prismatic form, a conical form or a pyramidal form, or a truncated conical form or a truncated pyramidal form.

The cell chip receiving part may include at least one connector connected to a liquid-type medium storage part.

The passage may have a zigzag form.

The passage may include a plurality of passages separated from each other in order to allow different kinds of liquid-type mediums to move independently therethrough.

According to another aspect of the present invention, there is provided a cell culture device, including: a cell chip receiving part having one surface provided with at least one passage through which a liquid-type medium is circulated; a cell chip coupled to the cell chip receiving part and having biomaterials attached thereto, the biomaterials being immersed in the passage; and a vortex generation part formed in the passage to generate a vortex in the liquid-type medium.

The cell chip may include a plurality of pillars having the biomaterials attached thereto and projected toward the passage.

The cell culture device may further include: a liquid-type medium storage part connected to the cell chip receiving part and storing the liquid-type medium supplied to the passage; and a circulator allowing the liquid-type medium to be circulated between the cell chip receiving part and the liquid-type medium storage part.

The liquid-type medium storage part may be divided into a plurality of liquid-type medium storage spaces in order to individually store the same or different types of liquid-type medium therein.

The circulator may be coupled to and separated from the plurality of liquid-type medium storage spaces and the cell chip receiving part so as to selectively connect the plurality of liquid-type medium storage spaces and the cell chip receiving part.

The circulator may include at least one pump.

At least one of the cell chip receiving part and the liquid-type medium storage part may include a filter filtering foreign objects included in the liquid-type medium circulating between the cell chip receiving part and the liquid-type medium storage part.

The vortex generation part may include a plurality of projections formed on a side wall of the passage.

The plurality of projections may be formed at intervals in a height direction of the side wall.

The plurality of projections may be formed to have different heights in a length direction of the side wall.

The vortex generation part may include a plurality of projections formed on a bottom of the passage.

The plurality of projections may be formed at intervals in a width direction of the bottom.

The plurality of projections may be formed to have different heights in a length direction of the bottom.

The vortex generation part may include two or more types of projections having different heights.

The vortex generation part may have a cylindrical form or a prismatic form, a conical form or a pyramidal form, or a truncated conical form or a truncated pyramidal form.

The cell chip receiving part may include at least one connector connected to a liquid-type medium storage part.

The passage may have a zigzag form.

The passage may include a plurality of passages separated from each other to independently move different kinds of liquid-type mediums.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view illustrating a cell culture device according to an embodiment of the present invention;

FIG. 2 is a cross-sectional view illustrating the cell culture device of FIG. 1, taken along line A-A;

FIGS. 3 and 4 are cross-sectional views respectively illustrating another form of the cell culture device of FIG. 1, taken along line A-A;

FIGS. 5 and 6 are plan views respectively illustrating another form of the cell culture device of FIG. 1;

FIG. 7 is a diagram illustrating a cell culture device according to another embodiment of the present invention;

FIGS. 8 through 10 are plan views respectively illustrating another form of a cell chip receiving part illustrated in FIG. 7;

FIG. 11 is a diagram illustrating a state in which a cell chip is mounted in the cell culture device of FIG. 7;

FIG. 12 is a cross-sectional view illustrating the cell culture device of FIG. 11, taken along line B-B;

FIG. 13 is an exploded perspective view illustrating a cell culture device according to another embodiment of the present invention;

FIG. 14 is an assembled perspective view of the cell culture device of FIG. 13; and

FIG. 15 is a diagram illustrating another form of a lower body illustrated in FIG. 13.

### DETAILED DESCRIPTION OF THE EMBODIMENTS

In vivo experiments may be performed to derive accurate reaction results between a liquid-type medium and biomaterials. Further, the in vivo experiments may be carried out by a method of dipping a cell chip having biomaterials attached thereto in a receiving part provided with a complicated liquid-type medium passage.

Meanwhile, a liquid-type medium may include a reagent, a culture medium, a drug or the like. The liquid-type medium may have two or more kinds of reaction reagents having different viscosities and densities. However, a passage through which the liquid-type medium is circulated may be considerably narrow, such that a mixture ratio of the reagents may be different in positions of the passage. That is, concentration deviations in a liquid-type medium may occur in a length direction of the passage or in a height direction of the passage. Concentration deviations in a liquid-type medium reduce reliability of a reaction experiment between biomaterials and the liquid-type medium, and therefore there is a need to develop a cell culture device capable of suppressing or minimizing the occurrence of concentration deviations in a liquid-type medium.

In order to solve the above defects of the present invention, a cell culture device generating a vortex in a liquid-type medium to minimize concentration deviations therein may be provided. For reference, in the present specification it is to be noted that the term vortex refers to an irregular flow provided to reduce concentration deviations in a liquid-type medium. Therefore, in the present specification, a vortex may include all flows other than a laminar flow.

Hereinafter, embodiments of the present invention will be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art . In the drawings, the shapes and dimensions of elements may be exaggerated for clarity, and the same reference numerals will be used throughout to designate the same or like elements.

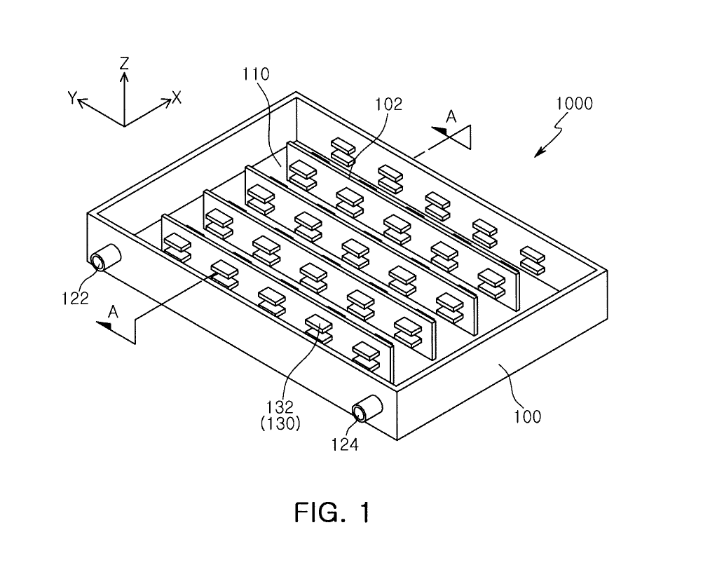

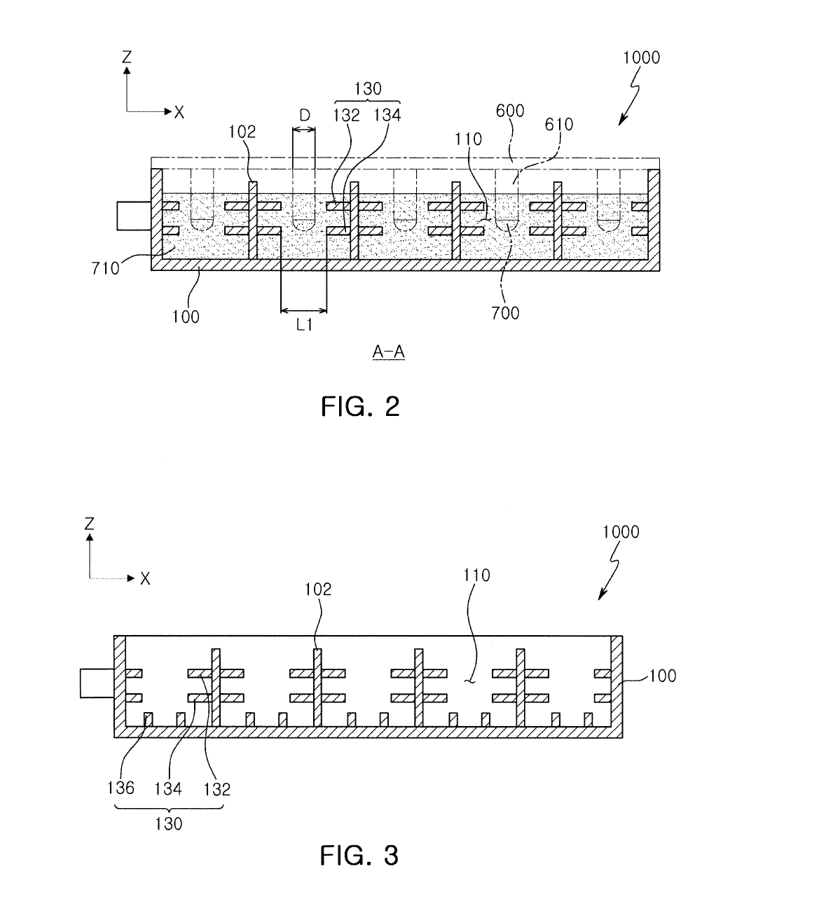

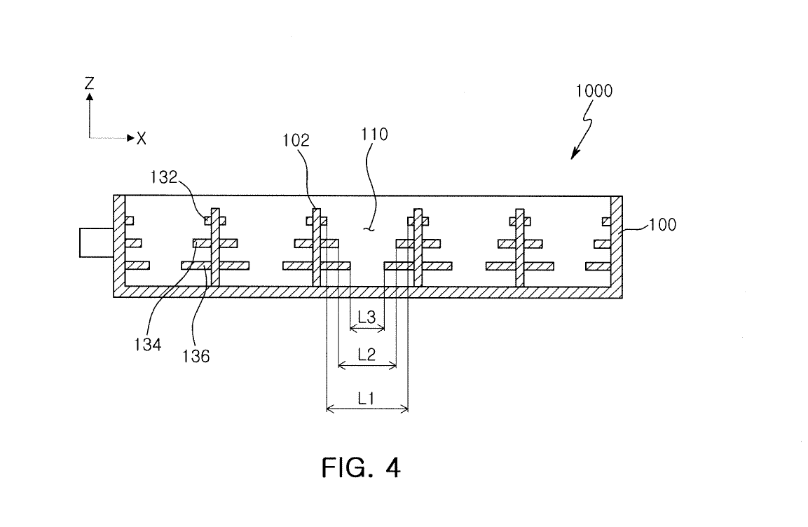

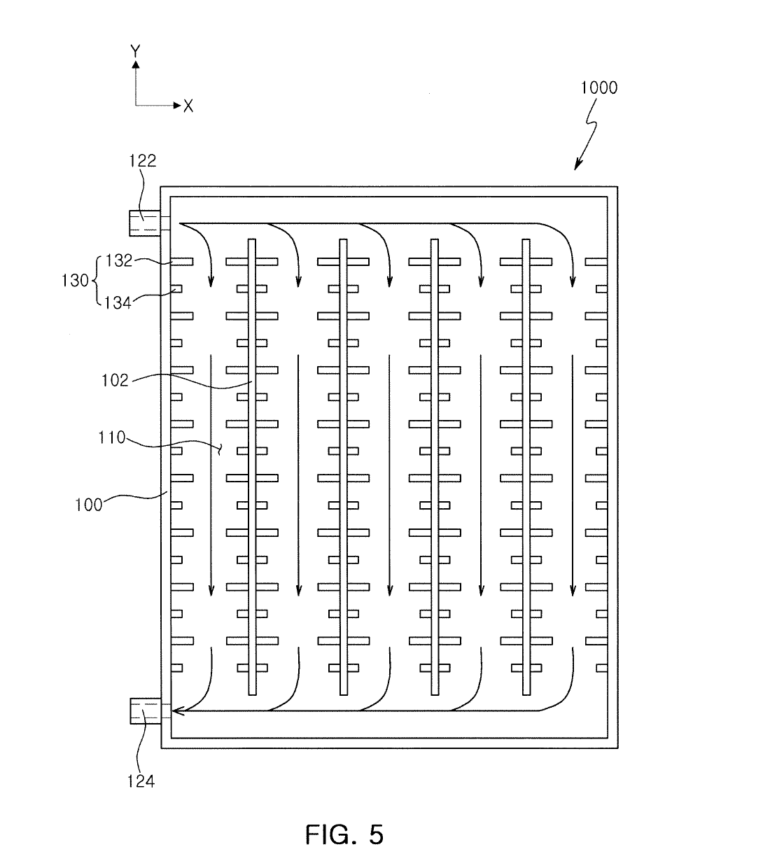

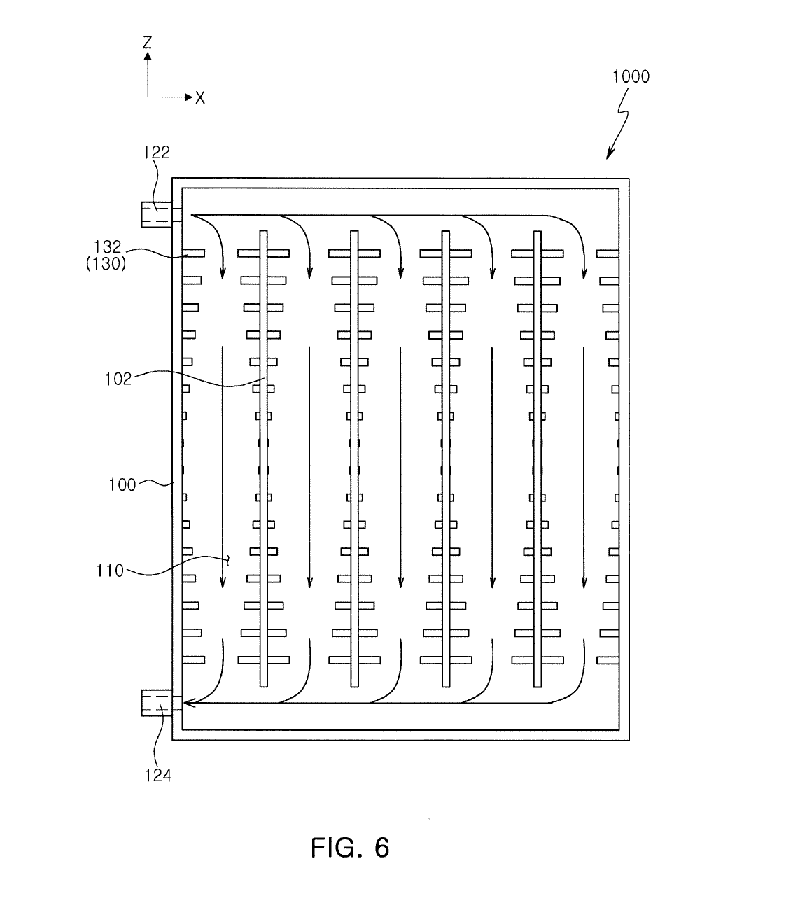

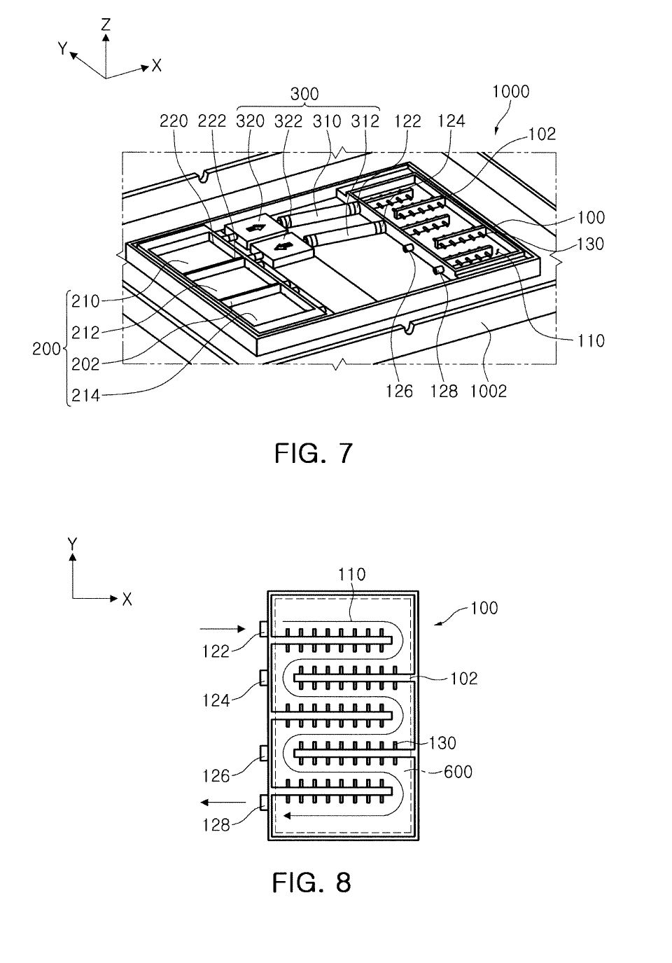

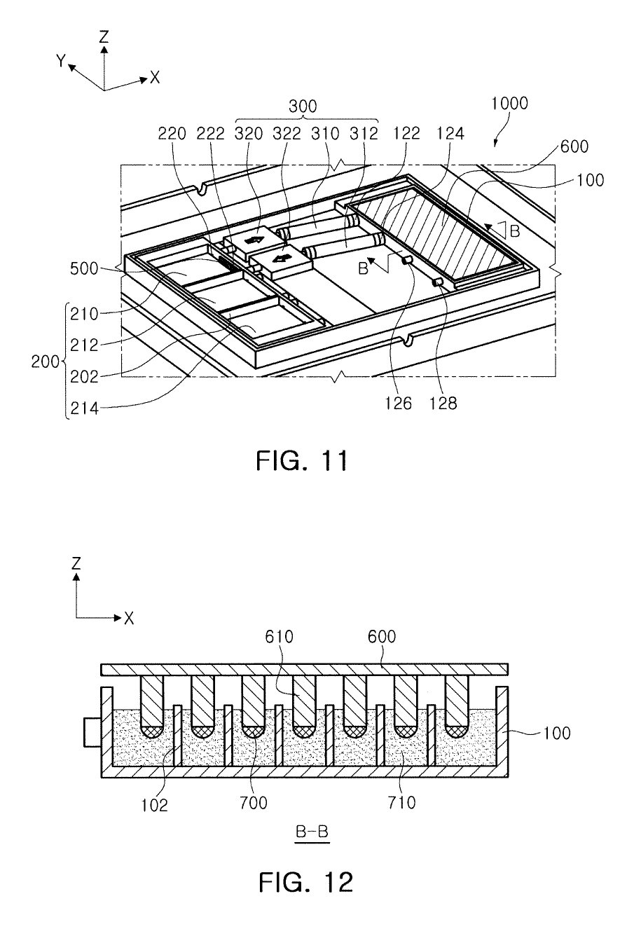

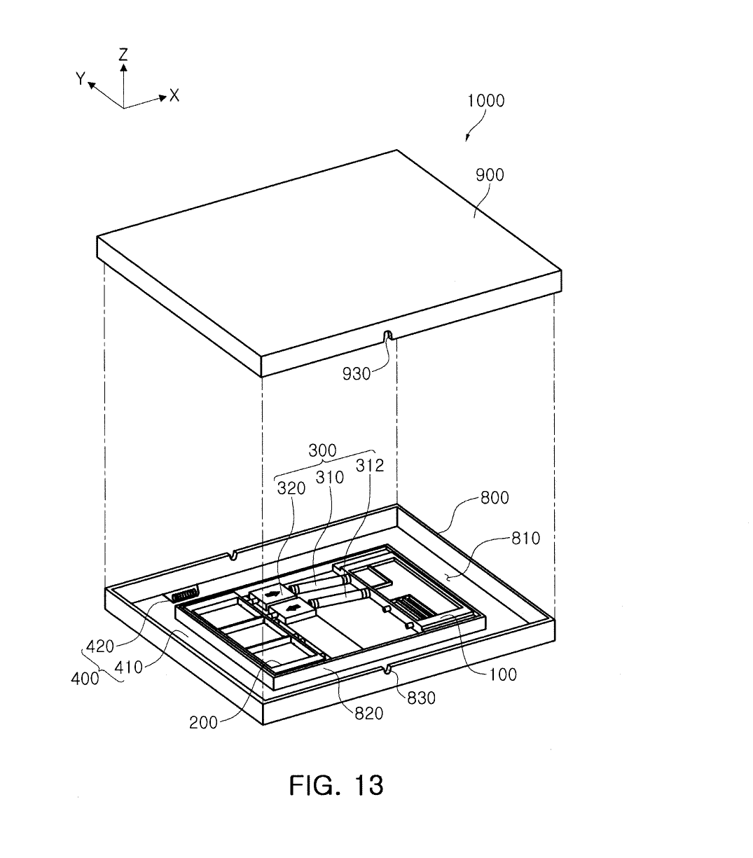

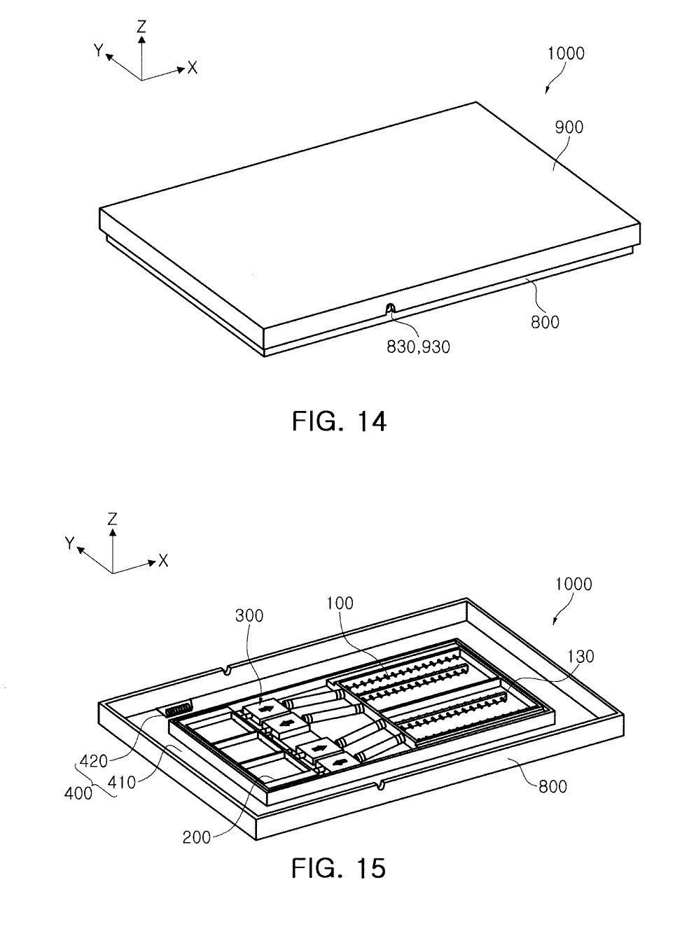

FIG. 1 is a perspective view illustrating a cell culture device according to an embodiment of the present invention. FIG. 2 is a cross-sectional view illustrating the cell culture device of FIG. 1, taken along line A-A. FIGS. 3 and 4 are cross-sectional views respectively illustrating another form of the cell culture device of FIG. 1, taken along line A-A. FIGS. 5 and 6 are plan views respectively illustrating another form of the cell culture device of FIG. 1. FIG. 7 is a diagram illustrating a cell culture device according to another embodiment of the present invention. FIGS. 8 through 10 are plan views respectively illustrating another form of a cell chip receiving part illustrated in FIG. 7. FIG. 11 is a diagram illustrating a state in which a cell chip is mounted in the cell culture device of FIG. 7. FIG. 12 is a cross-sectional view illustrating the cell culture device of FIG. 11, taken along line B-B. FIG. 13 is an exploded perspective view illustrating a cell culture device according to another embodiment of the present invention. FIG. 14 is an assembled perspective view of the cell culture device of FIG. 13. FIG. 15 is a diagram illustrating another form of a lower body illustrated in FIG. 13.

A cell culture device 1000 according to an embodiment of the present invention will hereinafter be described with reference to FIGS. 1 through 6.

The cell culture device 1000 according to the embodiment of the present invention may include a cell chip receiving part 100. In addition, the cell culture device 1000 may further include a liquid-type medium circulating part allowing a liquid-type medium to be continuously circulated in the cell chip receiving part 100.

The cell chip receiving part 100 may receive a liquid-type medium 710 and a cell chip 600. In other words, an inside of the cell chip receiving part 100 may be filled with the liquid-type medium 710 and the cell chip 600 may be disposed on one surface of the cell chip receiving part 100. Herein, the cell chip 600 may be disposed on one surface of the cell chip receiving part 100 while being vertically inverted as illustrated in FIG. 2 such that pillars 610 having biomaterials 700 attached thereto may be immersed in the liquid-type medium 710.

The cell chip receiving part 100 may be provided with partitions 102. The partitions 102 may be formed in the cell chip receiving part 100 in an optional form, such that at least one passage 110 through which the liquid-type medium 710 is circulated may be formed. For reference, in the present embodiment, a plurality of partitions 102 may be formed in parallel in one direction (the Y-axis direction in FIG. 1) of the cell chip receiving part 100 to form five passages 110.

The cell chip receiving part 100 may be provided with connectors 122 and 124. The connectors 122 and 124 may be connected to a liquid-type medium supplying part (for example, a liquid-type medium storage part 200 of FIG. 7) that supplies a liquid-type medium. The connectors 122 and 124 may be respectively disposed at both ends of the passage 110. In other words, the connector 122 may be disposed at one end of the passage 110 and the connector 124 may be disposed at the other end of the passage 110. In this case, the liquid-type medium 710 may be continuously circulated through the connector 122, the passage 110, and the connector 124.

A vortex generation part 130 alleviating concentration deviations in the liquid-type medium 710 may be formed in the passage 110. Here, the vortex generation part 130 may include a plurality of projections 132 and 134 formed on a side wall of the passage 110 as illustrated in FIG. 2. Alternatively, the vortex generation part 130 may include a plurality of projections 132, 134, and 136 formed on the side wall and the bottom of the passage 110 as illustrated in FIG. 3. The vortex generation part 130 so formed may continuously generate a vertical flow of the liquid-type medium 710 to maintain concentrations in the liquid-type medium 710 uniform over the whole of the cell chip receiving part 100. Therefore, according to the present embodiment, reliability of the reaction results between the biomaterials 700 and the liquid-type medium 710 may be improved.

Meanwhile, a distance L1 between the projections 132 and 134 facing each other may be greater than a diameter D of the pillar 610 formed in the cell chip 600 as illustrated in FIG. 2. This restriction may significantly reduce a phenomenon in which the biomaterials 700 contact the vortex generation part 130 during the coupling of the cell chip 600 and the cell chip receiving part 100.

The vortex generation part 130 may be modified in various manners as illustrated in FIGS. 3 through 6.

For example, the vortex generation part 130 may include the projections 132, 134, and 136 having different heights as illustrated in FIG. 4. In other words, the first projections 132 may be smaller than the second projections 134 and the second projections 134 may be smaller than the third projections 136. Therefore, the distance L1 between the first projections 132 may be larger than a distance L2 between the second projections 134 and the distance L2 between the second projections 134 may be larger than a distance L3 between the third projections 136. The structure of the vortex generation part 130 may facilitate vortex generations in the liquid-type medium as well as significantly reducing undesirable contact between the vortex generation part 130 and the biomaterials 700. For reference, the projections 132, 134, and 136 may have a cylindrical form or a prismatic form, a conical form or a pyramidal form, or a truncated conical form or a truncated pyramidal form.

The vortex generation part 130 may be configured to have another form in which the plurality of projections 132 and 134 are formed at predetermined intervals in the length direction (the Y-axis direction in FIG. 5) of the passage 110, as illustrated in FIG. 5. In addition, the vortex generation part 130 may include the projections 132 and 134 that are alternately disposed in the length direction of the passage 110 and have different heights. That is, a distance L4 between the projections 132 may be smaller than a distance L5 between the projections 134.

The vortex generation part 130 so formed may significantly reduce concentration deviations in the liquid-type medium that may occur in the length direction of the passage 110. Therefore, the vortex generation part 130 having such a form may be useful in the case of the passage 110 having a large length and a small width.

The vortex generation part 130 may be configured to have another form in which the projections 132 having lengths varied in the length direction (Y-axis direction in FIG. 6) of the passage 110 are formed, as illustrated in FIG. 6. Here, the projections 132 may be disposed in such a manner that the length thereof gradually decreases and then gradually increases in the length direction of the passage 110. Alternatively, the projections 132 may be locally formed in a portion of the cell chip receiving part 100, in which a flow velocity of the liquid-type medium 710 is low.

The vortex generation part 130 so configured may significantly reduce a flow resistance in the liquid-type medium due to the projections 132, such that a magnitude of driving force required to circulate the liquid-type medium may be reduced and current consumption may be reduced accordingly.

Next, the cell culture device 1000 according to another embodiment of the present invention will be described with reference to FIGS. 7 through 12.

The cell culture device 1000 may include the cell chip receiving part 100, the liquid-type medium storage part 200, and a circulator 300. In this configuration, the cell chip receiving part 100, the liquid-type medium storage part 200, and the circulator 300 may be integrally formed in a single body 1002. Alternatively, the cell chip receiving part 100, the liquid-type medium storage part 200, and the circulator 300 may be integrated in the single body. However, the cell chip receiving part 100, the liquid-type medium storage part 200, and the circulator 300 may not necessarily be formed integrally. For example, any one of the cell chip receiving part 100, the liquid-type medium storage part 200, and the circulator 300 may be separated therefrom.

The cell chip receiving part 100 may receive at least one cell chip. To this end, the cell chip receiving part 100 may include a space receiving the cell chip. The cell chip may be mounted in the space while being vertically inverted (see FIG. 12).

The cell chip receiving part 100 may include a plurality of first connectors 122, 124, 126, and 128 that are connected to the circulator 300. The first connectors 122, 124, 126, and 128 may be formed in one direction (the Y-axis direction in FIG. 7) of the cell chip receiving part 100. Here, formation positions of the first connectors 122, 124, 126, and 128 and formation intervals therebetween are not particularly limited. For example, the formation intervals between the first connectors 122, 124, 126, and 128 may be identical or may be partially different. Further, the number of first connectors 122, 124, 126, and 128 are not particularly limited. For example, FIG. 7 illustrates that four first connectors 122, 124, 126, and 128 are formed in the cell chip receiving part 100, but the number of first connectors may be increased and decreased if necessary.

Meanwhile, the cell chip receiving part 100 may have the passage 110 through which a flow of a liquid-type medium is induced. In other words, the passage 110 may be formed by the plurality of partitions 102 that partially partition the cell chip receiving part 100. The plurality of respective partitions 102 maybe provided with the vortex generation part 130.

The passage 110 so formed may lead the liquid-type medium introduced into the cell chip receiving part 100 to sequentially contact or react with at least one or more kinds of biomaterials attached to the cell chip.

Various forms of passage 110 will be described with reference to FIGS. 8 to 10.

According to a form of the passage 110, the passage 110 may have a zigzag form by the partitions 102 that extend in an X-axis direction, as illustrated in FIG. 8. In this case, the connectors 122 and 128 of the cell chip receiving part 100 may be connected to pipes 310 and 312, respectively, and the remaining connectors 124 and 126 may be closed. The passage 110 in this form may be appropriate for the case in which different kinds of biomaterials are arranged in a Y-axis direction.

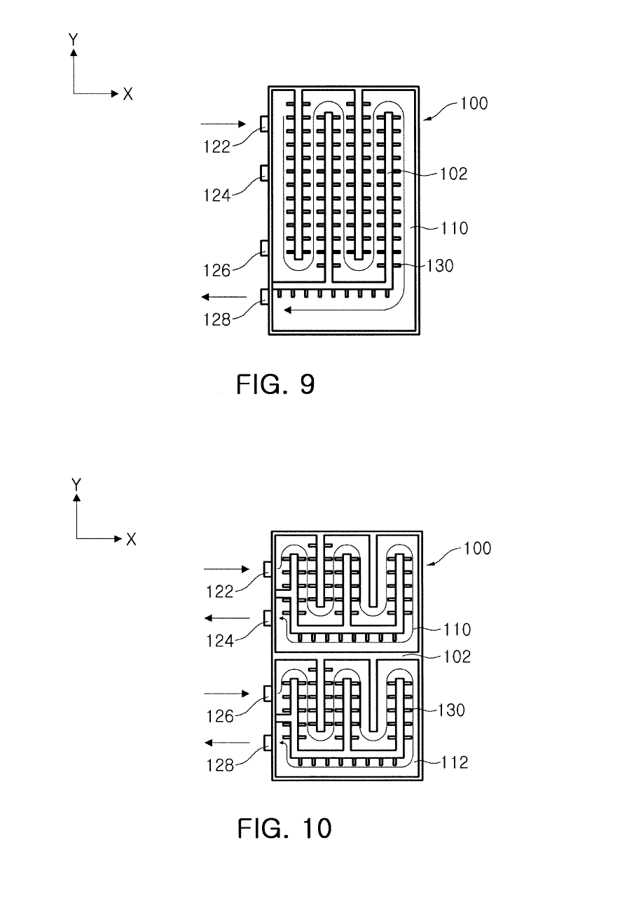

According to another form of the passage 110, the passage 110 may have a zigzag form by the partitions 102 that extend in a Y-axis direction, as illustrated in FIG. 9. In this case, any one of the connectors 122, 124, and 126 of the cell chip receiving part 100 may be connected to the first pipe 310 and the connector 128 may be connected to the second pipe 312. The passage 110 in the form may be appropriate for the case in which different kinds of biomaterials are arranged in an X-axis direction.

According to another form of passages 110 and 112, the passages 110 and 112 may be divided in two regions as illustrated in FIG. 10. In other words, the first passage 110 connected from the first connector 122 to the first connector 124 may be formed in one portion of the cell chip receiving part 100 and the second passage 112 connected from the first connector 126 to the first connector 128 may be formed in the remaining portion of the cell chip receiving part 100. The passages 110 and 112 so formed may be appropriate for the case in which different liquid-type mediums are experimented with regard to a single cell chip.

Meanwhile, the vortex generation part 130 may be formed in each passage 110 to actively generate the flow of the liquid-type medium, thereby suppressing the phenomenon in which a concentration in the liquid-type medium varies in the length direction of the passage 110 or the height direction of the passage 110.

The liquid-type medium storage part 200 may store the liquid-type medium therein. To this end, the liquid-type medium storage part 200 may include at least one liquid-type medium storage space, and in the embodiment, the liquid-type medium storage part 200 may include liquid-type medium storage spaces 210, 212, and 214. The liquid-type medium storage spaces 210, 212, and 214 may be divided by the partitions 202.

The same or different types of liquid-type medium may be stored in the liquid-type medium storage spaces 210, 212, and 214. For example, the first liquid-type medium storage space 210, the second liquid-type medium storage space 212, and the third liquid-type medium storage space 214 may store the same type of liquid-type medium. As another example, the first liquid-type medium storage space 210, the second liquid-type medium storage space 212, and the third liquid-type medium storage space 214 may respectively store different kinds of liquid-type mediums. As another example, the first liquid-type medium storage space 210 and the second liquid-type medium storage space 212 may each store the same type of liquid-type medium and the third liquid-type medium storage space 214 may store a different kind of liquid-type medium. However, the storage form of liquid-type medium is not limited to the foregoing three examples and may vary if necessary. Similarly, FIG. 7 illustrates that the liquid-type medium storage part 200 is divided into the three liquid-type medium storage spaces 210, 212, and 214, but the number of liquid-type medium storage spaces 210, 212, and 214 may be increased and decreased as needed.

The liquid-type medium storage part 200 may have a volume equal to or larger than that of the cell chip receiving part 100. For example, the liquid-type medium storage part 200 may have a sufficient volume such that a predetermined amount of liquid-type medium may be circulated between the cell chip receiving part 100 and the liquid-type medium storage part 200. Meanwhile, FIG. 7 illustrates that the volume of the liquid-type medium storage spaces 210, 212, and 214 is smaller than that of the cell chip receiving part 100, but the volume of the liquid-type medium storage spaces 210, 212, and 214 may be equal to or larger than that of the cell chip receiving part 100.

The liquid-type medium storage part 200 may include a plurality of second connectors 220 and 222 that are connected to the circulator 300. The second connectors 220 and 222 may be formed in one direction (the Y-axis direction in FIG. 7) of the liquid-type medium storage part 200. In other words, the second connectors 220 and 222 may be formed for each of the liquid-type medium storage spaces 210, 212, and 214. In this configuration, the second connector 220 maybe used as an outlet through which the liquid-type medium is discharged and the second connector 222 may be used as an inlet into which the discharged liquid-type medium is reintroduced. However, the outlet and the inlet are not defined in the second connectors 220 and 222 and the outlet and the inlet may be changed if necessary. For example, the second connector 222 may be the outlet and the second connector 220 may be the inlet.

The liquid-type medium storage part 200 may be connected to the cell chip receiving part 100 via the circulator 300. Therefore, the liquid-type medium of the drug storage part 200 may be supplied to the cell chip receiving part 100 via the circulator 300. Similarly, the liquid-type medium supplied to the cell chip receiving part 100 may be provided to the liquid-type medium storage part 200 via the circulator 300.

The liquid-type medium storage part 200 configured as above maybe disposed to face the cell chip receiving part 100. For example, the liquid-type medium storage part 200 may be disposed symmetrically with regard to the cell chip receiving part 100 based on a Y axis, as illustrated in FIG. 7. However, the disposition structure of the liquid-type medium storage part 200 is not limited to the form illustrated in FIG. 7, but may vary as needed.

The circulator 300 may be disposed between the cell chip receiving part 100 and the liquid-type medium storage part 200. In other words, the circulator 300 may connect the first connection pipes 122, 124, 126, and 128 of the cell chip receiving part 100 to the second connectors 220 and 222 of the liquid-type medium storage part 200. In addition, the circulator 300 may transfer the liquid-type medium of the liquid-type medium storage part 200 to the cell chip receiving part 100 and may introduce the liquid-type medium transferred to the cell chip receiving part 100 into the liquid-type medium storage part 200. Therefore, the liquid-type medium of the liquid-type medium storage part 200 may be circulated between the liquid-type medium storage part 200 and the cell chip receiving part 100 via the circulator 300.

The circulator 300 may include the pipes 310 and 312. In other words, the first pipe 310 may connect the first connector 122 of the cell chip receiving part 100 to the second connector 220 of the liquid-type medium storage part 200 and the second pipe 312 may connect the first connector 124 of the cell chip receiving part 100 to the second connector 222 of the liquid-type medium storage part 200. However, the connection form is only an example, and therefore may be changed if necessary. For example, the first pipe 310 may connect the first connector 122 of the cell chip receiving part 100 to the second connector 220 of the liquid-type medium storage part 200 and the second pipe 312 may connect the first connector 128 of the cell chip receiving part 100 to the second connector 222 of the liquid-type medium storage part 200. In addition, FIG. 7 illustrates that the pipes 310 and 312 connect the cell chip receiving part 100 to the first liquid-type medium storage space 210, but the pipes 310 and 312 may connect the cell chip receiving part 100 to the second liquid-type medium storage space 212 or the third liquid-type medium storage space 214 if necessary.

The circulator 300 may further include pumps 320 and 322. The pumps 320 may be coupled to the pipes 310 and 312 or the connectors 122, 124, 126, 128, 220, and 222 to transfer the liquid-type medium. For example, the first pump 320 coupled to the first pipe 310 may pump the liquid-type medium in the liquid-type medium storage part 200 to the cell chip receiving part 100 and the second pump 322 coupled to the second pipe 312 may pump the liquid-type medium in the cell chip receiving part 100 to the liquid-type medium storage part 200. Meanwhile, the present embodiment illustrates that the pumps 320 and 322 are respectively coupled to the first pipe 310 and the second pipe 312, but the first pump 320 or the second pump 322 may be omitted if necessary.

Meanwhile, the circulator 300 may be freely coupled to and separated from the cell chip receiving part 100 and the liquid-type medium storage part 200. Therefore, a user may select any one of the plurality of liquid-type medium storage spaces 210, 212, and 214 according to the experimental goal of an experiment using the cell chip mounted in the cell chip receiving part 100.

As illustrated in FIG. 11, in the cell culture device 1000, the at least one cell chip 600 may be received in the cell chip receiving part 100. Here, as illustrated in FIG. 12, the cell chip 600 may be disposed in a vertically inverted state. Therefore, the biomaterials 700 attached to the pillars 610 of the cell chip 600 may react with the liquid-type medium 710 supplied to the cell chip receiving part 100. Here, the liquid-type medium 710 is continuously circulated between the cell chip receiving part 100 and the liquid-type medium storage part 200 via the circulator 300, such that the reaction of the biomaterials 700 to the liquid-type medium 710 may be observed in an environment similar to that of the interior of a body and the long-term experiment or observation of the biomaterials 700 to the liquid-type medium 710 may be performed.

Further, in the cell culture device 1000 according to the embodiment of the present invention, various forms of the passage 110 may be formed in the cell chip receiving part 100, such that an environment similar to the conditions of an in vivo experiment or an in vivo culture may be created. Therefore, the cell culture device 1000 according to the embodiment of the present invention may allow for relatively accurate observations of an effect of the liquid-type medium 710 on a human body.

In addition, the cell culture device 1000 according to the embodiment of the present invention may allow for an observation of reactions of the biomaterials 700 to an accumulation of waste products in response to the continuous supply of a liquid-type medium and concentration changes in the liquid-type medium.

Meanwhile, in the cell culture device 1000 according to the embodiment of the present invention, a filter may be mounted in the cell chip receiving part 100 or the liquid-type medium storage part 200 to selectively remove foreign objects occurring during the reaction process between the liquid-type medium 710 and the biomaterials 700. In addition, the foreign objects or the waste product occurring during the reaction process of the liquid-type medium 710 and the biomaterials 700 may be separately observed by collecting the foreign objects filtered by the filter 500.

Next, the cell culture device 1000 according to another embodiment of the present invention will be described with reference to FIGS. 13 through 15. For reference, in the present embodiment, the same reference numerals will be used to describe the same components as those of another embodiment. In addition, a detailed description of these components will be omitted.

The cell culture device 1000 according to the present embodiment may further include a lower body 800 and an upper body 900.

The lower body 800 may include a receiving space 810. The receiving space 810 may be provided with a main wall part 820 that separates a central portion thereof from an edge portion thereof. Therefore, the receiving space 810 may be divided into two portions by the main wall part 820. In this configuration, a space (a first space) enclosed by the main wall 820 may accommodate all of the cell chip receiving part 100, the liquid-type medium storage part 200, and the circulator 300. In addition, the outer side (a second space) separated by the main wall part 820 from the first space may be provided with a humidity control part 400.

The humidity control part 400 may include a water storage space 410 and a heater 420. The water storage space 410 may store a considerable amount of water and the heater 420 may heat the water stored in the water storage space 410. The humidity control part 400 so configured generates vapor around the main wall part 820 to constantly maintain the humidity in the interior of the cell culture device 1000. Meanwhile, FIGS. 13 and 14 illustrate that the heater 420 is formed in a portion of the water storage space 410; however the heater 420 may be formed over the entire region of the water storage space 410 if necessary. In addition, the heater 420 may be omitted if necessary.

The upper body 900 may be coupled to the lower body 800. In other words, the upper body 900 may be coupled to the lower body 800 to close the receiving space 810. The upper body 900 so configured blocks foreign objects from being introduced into the cell culture device 1000 from the outside and protects the cell chip receiving part 100 and the liquid-type medium storage part 200 from external impacts.

The lower body 800 and the upper body 900 may be provided with holes 830 and 930, respectively. An electrical wire (not illustrated) connecting the pumps 320 and 322 to the external power supply may be drawn out through the holes 830 and 930. However, the holes 830 and 930 may not only be used as holes through which the electric wire is drawn out but may be used as an air path if necessary.

As set forth above, according to the embodiment of the present invention, the reaction between a liquid-type medium and cells can be observed or inspected under the environment similar to the interior of the body.

Therefore, according to the embodiment of the present invention, the reliability of experiment results regarding the reaction between the liquid-type medium and cells can be increased.

While the present invention has been shown and described in connection with the embodiments, it will be apparent to those skilled in the art that modifications and variations can be made without departing from the spirit and scope of the invention as defined by the appended claims.

What is claimed is:

1. A cell culture device, comprising:
a cell chip receiving part coupled to a cell chip and having at least one passage through which a liquid-type medium is circulated; and
a vortex generation part formed in the passage to generate a vortex in the liquid-type medium.

2. The cell culture device of claim 1, wherein the vortex generation part includes a plurality of projections formed on a side wall of the passage.

3. The cell culture device of claim 2, wherein the plurality of projections are formed at intervals in a height direction of the side wall.

4. The cell culture device of claim 2, wherein the plurality of projections are formed to have different heights in a length direction of the side wall.

5. The cell culture device of claim 1, wherein the vortex generation part includes a plurality of projections formed on a bottom of the passage.

6. The cell culture device of claim 5, wherein the plurality of projections are formed at intervals in a width direction of the bottom.

7. The cell culture device of claim 5, wherein the plurality of projections are formed to have different heights in a length direction of the bottom.

8. The cell culture device of claim 1, wherein the vortex generation part includes two or more types of projections having different heights.

9. The cell culture device of claim 1, wherein the vortex generation part has a cylindrical form or a prismatic form, a conical form or a pyramidal form, or a truncated conical form or a truncated pyramidal form.

10. The cell culture device of claim 1, wherein the cell chip receiving part includes at least one connector connected to a liquid-type medium storage part.

11. The cell culture device of claim 1, wherein the passage has a zigzag form.

12. The cell culture device of claim 1, wherein the passage includes a plurality of passages separated from each other in order to allow different kinds of liquid-type mediums to move independently therethrough.

13. A cell culture device, comprising:
a cell chip receiving part having one surface provided with at least one passage through which a liquid-type medium is circulated;
a cell chip coupled to the cell chip receiving part and having biomaterials attached thereto, the biomaterials being immersed in the passage; and
a vortex generation part formed in the passage to generate a vortex in the liquid-type medium.

14. The cell culture device of claim 13, wherein the cell chip includes a plurality of pillars having the biomaterials attached thereto and projected toward the passage.

15. The cell culture device of claim 13, further comprising:
a liquid-type medium storage part connected to the cell chip receiving part and storing the liquid-type medium supplied to the passage; and
a circulator allowing the liquid-type medium to be circulated between the cell chip receiving part and the liquid-type medium storage part.

16. The cell culture device of claim 15, wherein the liquid-type medium storage part is divided into a plurality of liquid-type medium storage spaces in order to individually store the same or different types of liquid-type medium therein.

17. The cell culture device of claim 16, wherein the circulator is coupled to and separated from the plurality of liquid-type medium storage spaces and the cell chip receiving part so as to selectively connect the plurality of liquid-type medium storage spaces and the cell chip receiving part.

18. The cell culture device of claim 15, wherein the circulator includes at least one pump.

19. The cell culture device of claim 15, wherein at least one of the cell chip receiving part and the liquid-type medium storage part includes a filter filtering foreign objects included in the liquid-type medium circulating between the cell chip receiving part and the liquid-type medium storage part.

20. The cell culture device of claim 13, wherein the vortex generation part includes a plurality of projections formed on a side wall of the passage.

21. The cell culture device of claim 20, wherein the plurality of projections are formed at intervals in a height direction of the side wall.

22. The cell culture device of claim 20, wherein the plurality of projections are formed to have different heights in a length direction of the side wall.

23. The cell culture device of claim 13, wherein the vortex generation part includes a plurality of projections formed on a bottom of the passage.

24. The cell culture device of claim 23, wherein the plurality of projections are formed at intervals in a width direction of the bottom.

25. The cell culture device of claim 23, wherein the plurality of projections are formed to have different heights in a length direction of the bottom.

26. The cell culture device of claim 13, wherein the vortex generation part includes two or more types of projections having different heights.

27. The cell culture device of claim 13, wherein the vortex generation part has a cylindrical form or a prismatic form, a conical form or a pyramidal form, or a truncated conical form or a truncated pyramidal form.

28. The cell culture device of claim 13, wherein the cell chip receiving part includes at least one connector connected to a liquid-type medium storage part.

29. The cell culture device of claim 13, wherein the passage has a zigzag form.

30. The cell culture device of claim 13, wherein the passage includes a plurality of passages separated from each other to independently move different kinds of liquid-type mediums.
